(19) United States
(12) Patent Application Publication
(10) Pub. No.: US 2012/0064595 A1
(43) Pub. Date: Mar. 15, 2012

(54) Method for accumulating cells

(75) Inventors: Shogo Miyata, Yokohama-shi (JP); Shinya Takeuchi, Kyoto-shi (JP)

(73) Assignees: NISSHA PRINTING CO., LTD., Kyoto-shi (JP); KEIO UNIVERSITY, Minato-ku (JP)

(21) Appl. No.: 13/067,743

(22) Filed: Jun. 23, 2011

(30) Foreign Application Priority Data
Sep. 14, 2010 (JP) 2010-204989

(51) Int. Cl. C12N 13/00

(52) U.S. Cl. 435/173.9

(57) ABSTRACT

The present invention has as an object to simplify the implant work in a series of the process from the separation of target cells by dielectrophresis to the cell cultivation and to realize a cell accumulation method that prevents bacteria contamination by bacteria.

Method to achieve the objects

The cell accumulation device includes the accumulation and cultivation vessel having a first electrode unit 16, an aggregation of the plural number of individual electrodes, allocated on the first substrate and a second electrode unit 17 allocated on the second substrate. The cell accumulation method has 1) a microbead trapping process, wherein microbeads 2 having biocompatibility are sent to the accumulation and cultivation vessel and AC voltage is applied to the first electrode unit and the second electrode unit to trap the microbeads on the individual electrodes and 2) a cell collection process, wherein after the microbead trapping process cellular suspension is sent into the inside of the vessel 101 of the accumulation and cultivation vessel 1 and AC voltage is applied to the first electrode unit and the second electrode unit to collect active cells 41 on said trapped microbeads.

### TECHNICAL FIELD

The present invention relates to a method for accumulating cells on microbeads having biocompatibility by using dielectrophresis.

### BACKGROUND ART

Because different cells have different dielectrophoretic characteristics according to their type, techniques for separating target cells in cellular suspension having a mixture of two or more kinds of cells by using dielectrophoresis are known.

One known arrangement is to apply AC voltage with a specific frequency to a pair of electrodes with a shape similar to alternate combs allocated in a separation vessel to form an unequal AC electric field between the electrodes placed opposite each other. The target cells in the cellular suspension placed in the unequal AC electric field are drawn to the electrode on one side by dielectrophresis and therefore are separated (refer to patent document 1 for instance).

### DOCUMENTS OF THE PRIOR ART

### Patent Document

Patent document 1: Japanese Patent Laid-Open No. 2008-263847 bulletin

### SUMMARY OF THE INVENTION

### Objects to be Solved by the Invention

In order to utilize the cell separation technique described above for regenerative medicine, particularly drug delivery system, it is necessary to obtain a large number of said cells by subculturing separated cells repeatedly.

In the prior art, cells collected on an electrode surface in a separation vessel after the dielectrophresis operation are taken from the separation vessel and then implanted in a culture vessel such as test tube and petri dish for subculture.

However, the prior art procedures have the problem that the implant work causes contamination by bacteria, which results in a reduction in efficiency of cell cultivation.

In view of the above problem in the prior art, the present invention has as an object to simplify the implant work in a series of the process from the separation of target cells to the cell cultivation and to realize a cell accumulation method that prevents contamination by bacteria.

### Method to Achieve the Objects

To achieve the objects, the cell accumulation method of the present invention is a cell accumulation method comprising

a cell accumulation device comprising
an accumulation and cultivation vessel with an inlet and an outlet, surrounded by a first substrate, a second substrate allocated against the first substrate, and the sidewall, having a first electrode unit, composed of the plural number of individual electrodes exposed on the inside of the accumulation and cultivation vessel, allocated on the first substrate and a second electrode unit allocated on the second substrate, and
a power supply unit applying AC voltage between the first electrode and the second electrode, and
a cell accumulation method comprising the following processes a) and b) which method utilizes dielectrophoretic microbeads having biocompatibility;
   a) a microbead trapping process, where a microbead having biocompatibility is sent to said accumulation and cultivation vessel from said inlet and AC voltage is applied to the first electrode unit and the second electrode unit by said power supply unit to trap said microbeads on the individual electrodes, and
   b) a cell collection process, where after the microbead trapping process cellular suspension containing collection target cells is sent into said accumulation and cultivation vessel from said inlet and AC voltage is applied to the first electrode unit and the second electrode unit by said power supply unit to collect said collection target cells on said trapped microbeads.

In the present invention, the cellular suspension used in the cell collection process (process b) just has to contain the collection target cells. The following are some examples of the cellular suspension:

(1) mixture of collection target cells, one or more kinds of other cells, and buffer solution; and
(2) mixture of collection target cells and buffer solution.

The present invention is to collect the collection target cells on microbeads. Therefore, the cell collection process (process b) is performed with the intention to

(1) separate the collection target cells from two or more kinds of cells;
(2) concentrate the collection target cells from subtle suspension; and
(3) purify the collection target cells in the suspension containing impurities.

In a preferred embodiment of the present invention, said microbead having biocompatibility may be a bead comprising collagen and alginate.

In another preferred embodiment of the present invention, the first electrode unit may have said individual electrodes, where an electrode member having a flat surface shape is formed on the surface of the first substrate, an insulated layer comprising insulating material is formed on the surface of said electrode member, and said insulated layer is removed partly in a circle.

In still another preferred embodiment of the present invention, the first electrode unit may have said individual electrodes, where a conducting layer of said electrode member is formed on the surface of the first substrate, said insulated layer is formed on said conducting layer, and said insulated layer of said individual electrode part is removed by irradiation of a laser, which method is based on the laser-etching method.

### Effectiveness of the Invention

In addition to other features, the cell accumulation method of the present invention provides trapping of the microbeads having biocompatibility on the individual electrodes in the accumulation and cultivation vessel and collecting of cells on the microbeads. The microbead becomes a scaffold material for cultivating the cells. Therefore, this cell accumulation method requires no implantation of collected cells and allows cultivation of cells in the accumulation and cultivation vessel, where the cell accumulation process has already finished. This prevents contamination by bacteria during the implantation process.

The trapping of said microbeads is performed by dielectrophresis. The power supply unit, a component of a cell separation unit using a conventional dielectrophoresis, may be used as a power supply unit for the cell accumulation device with no major changes being made.

### BRIEF DESCRIPTION OF THE DRAWINGS

FIG. 1 is an explanation drawing of the cell accumulation method of the present invention. FIG. 1(a) shows the microbead trapping process in progress, and FIG. 1(b) shows the microbead trapping process that came to an end. FIG. 1(c) shows the cell collecting process in progress, and FIG. 1(d) shows the cell collecting process that came to an end. Each of the four (4) figures shows a cross-section drawing of the accumulation and cultivation vessel.

FIG. 2 is a perspective view of the cell accumulation device, where a part of the second substrate 12 is cut to show the first electrode 16.

FIG. 3 is a plane view of the accumulation and cultivation vessel 10, where the second substrate 12 is cut and removed from said vessel to show the view of the first substrate from the side of the second substrate.

FIG. 4 is a cross-sectional view of the accumulation and cultivation vessel 10, where said vessel is cut along the plane surface indicated by the arrow A and the arrow B.

FIG. 5 is an explanation drawing of a device used for producing collagen microbeads.

### EMBODIMENTS TO CARRY OUT THE INVENTION

With reference to the drawings, a description will be made of the cell accumulation method according to the embodiment of the present invention. In the drawings referred to in the specification, some of the composition elements are shown exaggeratedly in schematic form to make it easy to understand the present invention. Therefore, some of the dimensions and ratios between the composition elements are different from the real ones. In addition, measurements, materials, shapes, relative positions, etc. of the members and parts described in the embodiments of the present invention are merely examples and are not intended to restrict the scope of the present invention, except as specifically described.

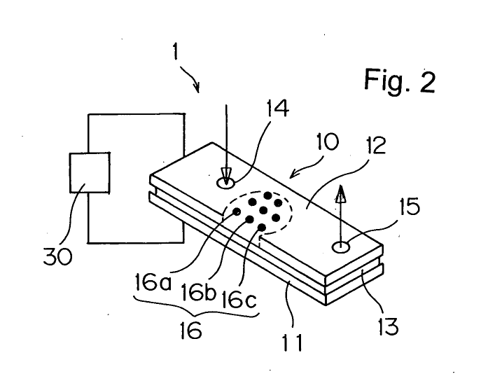

The cell accumulation method of the present invention uses the cell accumulation device 1. Referring to FIG. 2, the cell accumulation device 1 comprises the accumulation and cultivation vessel 10 and the power supply unit 30. In the accumulation and cultivation vessel 10, the first substrate 11, flat plate-shaped, and the second substrate 12, flat plate-shaped, are allocated just like two flat plates face each other, and the sidewall material 13 is placed between the first substrate 11 and the second substrate 12.

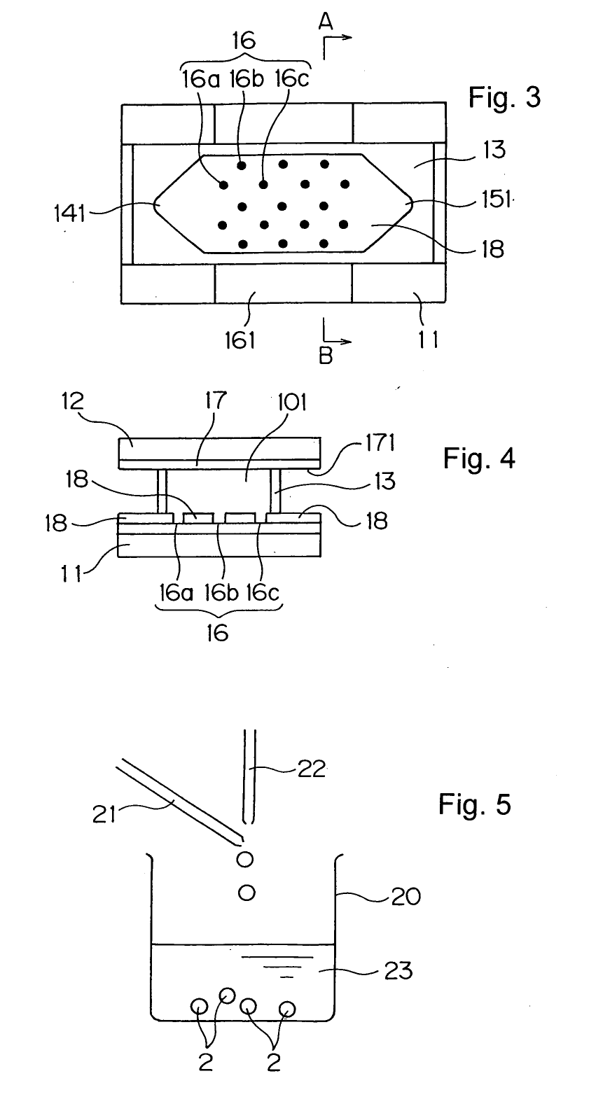

Referring to FIG. 3, a plane view of the accumulation and cultivation vessel 10, where the second substrate 12 is cut and removed from the accumulation and cultivation vessel 10 to show the view of the first substrate from the second substrate, and FIG. 4, a cross-sectional view of the accumulation and cultivation vessel 10, where the vessel is cut across the plane surface indicated by the arrow A and the arrow B, collectively, the sidewall material 13 is a frame-shaped board material with a window unit. Said window unit constitutes the inside of the vessel 101 of the accumulation and cultivation vessel 10. In other words, the inside of the vessel 101 is a space surrounded by the first substrate 11, the second substrate 12, and the window sidewall of the sidewall material 13.

The second substrate 12 has two (2) holes that lead to the inside of the vessel 101. One of the holes is the inlet 14 and the other is the outlet 15. The liquid that came into the inside of the vessel 101 from the inlet 14 drains out from the outlet 15. FIG. 3 shows the inlet opposite part 141 that faces the inlet 14 and the outlet opposite part 151 that faces the outlet 15.

A first on-off valve, a tube, a pump, etc. needed for sending solution (these are not shown in the figures) are connected to the inlet 14, if necessary. A second on-off valve, a tube, etc. (these are not shown in the figures) are connected to the outlet 15, if necessary. As described below, because the accumulation and cultivation vessel 10 is used as a vessel for cell culture, it is preferable that the tube and the pump connected upstream of the first on-off valve and the tube connected downstream of the second on-off valve be demountable.

The inlet 14 and the outlet 15 may be attached to the first substrate. As an alternative, either of the inlet 14 and the outlet 15 may be attached to the first substrate and the other may be attached to the second substrate.

The first electrode 16 is allocated on the upper surface of the first substrate 11 (i.e., the surface contacting the inside of the vessel 101). The first electrode unit 16 comprises a plurality of individual electrodes 16a, 16b, 16c, . . . . A plurality of individual electrodes 16a, 16b, 16c, . . . are allocated with an equal distance each other. In this embodiment, a plurality of individual electrodes 16a, 16b, 16c, . . . , which are circular, are allocated so that the center of each individual electrode is at the corner of a latticed square with a distance of 550 μm between lattices. FIG. 2, in which a part of the second substrate 12 is cut, shows the first electrode 16.

There is no restriction on the shape of the flat surface of the individual electrodes 16a, 16b, 16c, . . . ; however, a circular form is preferable. If the individual electrodes 16a, 16b, 16c, . . . are circular, each density of the lines of electric force that appear on the individual electrodes at the time of AC voltage being applied is constant, which allows the density of the lines of electric force on the microbead having biocompatibility trapped on the individual electrode also to be constant. Therefore, when the cellular suspension flows into the cell accumulation device, the target cells in the cellular suspension are collected uniformly at the surface of the microbead without gathering together on a specific part of the microbead. Therefore, any collection target cell collected as described above can grow on the microbeads having a biocompatibility as scaffold materials.

The individual electrodes 16a, 16b, 16c, . . . may not necessarily be allocated with an equal distance each other. Even if the individual electrodes 16a, 16b, 16c, . . . are allocated with an unequal distance each other, the microbeads having a biocompatibility can be trapped on the individual electrodes by dielectrophresis and the collection target cells can be collected on such microbeads trapped on the electrodes. However, it is preferable that the individual electrodes 16a, 16b, 16c, . . . be allocated with an equal distance each other, partly because any of the cells that are on the individual electrodes at the time of the cell cultivation can equally metabolize nutrients that exist around.

In the first electrode unit 16, a conducting layer is formed on the surface of the first substrate 11 and non-individual electrode areas of said conducting layer are coated with the insulating layer 18. Said conducting layer is extended to the area protruding from the internal portion of the vessel of the first substrate. The protruded area is the terminal unit 161 that is conducted to the first electrode unit 16.

Preferably, the electrode unit 16 is formed by using photolithography or laser etching.

If the electrode unit 16 is formed by using photolithography, FTO (fluorine doped tin oxide) film, which serves as a conducting layer, is formed on one side of the material, e.g., glass, of the first substrate. After that, photo-resist film, which serves as an insulating layer, is applied to the FTO film. Then, the photo-resist film is exposed and patterned with the individual electrodes to remove the photo-resist film on the individual electrode part. The use of photolithography permits the high-resolution formation of the electrode unit 16. Therefore, the microbeads having biocompatibility described below can be trapped on the electrode unit 16, with high accuracy of position.

If the first electrode unit 16 is formed by using laser etching, just like the method described above, FTO (fluorine doped tin oxide) film is formed on one side of material of the first substrate. After that, an insulating layer is applied to the FTO film. Then, the insulating layer is exposed with laser light and patterned with the individual electrodes to remove the insulating layer on the individual electrode part. The laser used for laser etching may be carbon dioxide, YAG, ruby or YVO4.

When laser etching is used, any material may be selected for the insulating layer. For example, the insulating layer may be formed by using the waterproof material, if desired. The use of the waterproof material prevents the insulating layer from peeling from the conducting layer such as FTO film, which extends the useful life of the first electrode unit. Furthermore, laser etching needs fewer processes for the formation of the first electrode unit than other methods including photolithography described above, which offers an economic advantage.

The second electrode unit 17 is allocated on the under surface of the second substrate 12 (i.e., the surface contacting the inside of the vessel 101). The second electrode unit is formed on the whole surface of the inside of the vessel laid off by the second substrate 12. The second electrode unit 17 is the conducting layer that was formed on the surface of the second substrate 12. Said conducting layer is extended to the area protruding from the internal portion of the vessel of the second substrate. The protruded area is the terminal unit 171 that is conducted to the first electrode unit 17.

The first substrate 11 and the second substrate 12 may be made of glass, acrylic plastic or any other material.

The first electrode unit 16 and the second electrode unit 17 may be made of transparent conducting layers (FTO, ITO (indium tin oxide), tin oxide, etc.), deposited metal film, or any other material. Considering that the accumulation and cultivation vessel 10 is used as a cell culture vessel, it is preferable that the second substrate 12 be made of glass and the second electrode unit 17 be of transparent conducting layer, from the viewpoint of easy visual observation of the inside of the vessel, easy wash of the vessel, easy sterilization, etc.

The sidewall material 13 may be made of silicon, for example.

The power supply unit 30 of the cell collection device 1 is a unit that applies AC voltage to the first electrode unit 16 and the second electrode unit 17. The power supply unit 30 provides a variable AC frequency and a variable voltage. The output lead of the power supply unit 30 is connected to the terminal 161 and the terminal 171. Because the accumulation and cultivation vessel 10 is used as a vessel for cell culture, it is preferable that the connection of the output lead of the power supply unit 30 to the terminal 161 and to the terminal 171 be demountable, by using a clip, a plug, etc.

Next, a description shall be provided for the way to make collagen microbeads that are microbeads having biocompatibility. Collagen/alginate mixed solution containing 10 mg/ml of collagen powder and 2% of alginate sodium is prepared. Furthermore, 102 mM of CaCl₂ solution is prepared as gel solution.

FIG. 5 is an explanation drawing of a device used for producing collagen microbeads. The gel solution 23 described above is reserved in the bead making vessel 20. Using a syringe pump (not shown in figures), the mixed solution described above is pushed out through the material pushing out nozzle 21. At that time, gas is sprayed onto the material pushing out nozzle 21 from the gas nozzle 22 in order for the droplets of the mixed solution to have about the same size. Preferably the gas from the gas nozzle is an inert gas. Therefore, in this embodiment nitrogen gas was used. In this way, collagen microbeads with the nearly-constant particle size were made.

Microbeads consisting only of collagen do not provide dielectrophoresis phenomenon; however, collagen microbeads to which alginate is added provide dielectrophoresis phenomenon. The collagen microbead is a microbead having biocompatibility and becomes a scaffold material for cultivating cells. The collagen microbead is suitable for cultivating animal-derived cells.

Other examples of microbead having biocompatibility are an agarose bead and an alginate bead. An alginate bead is suitable for cultivating plant-derived cells.

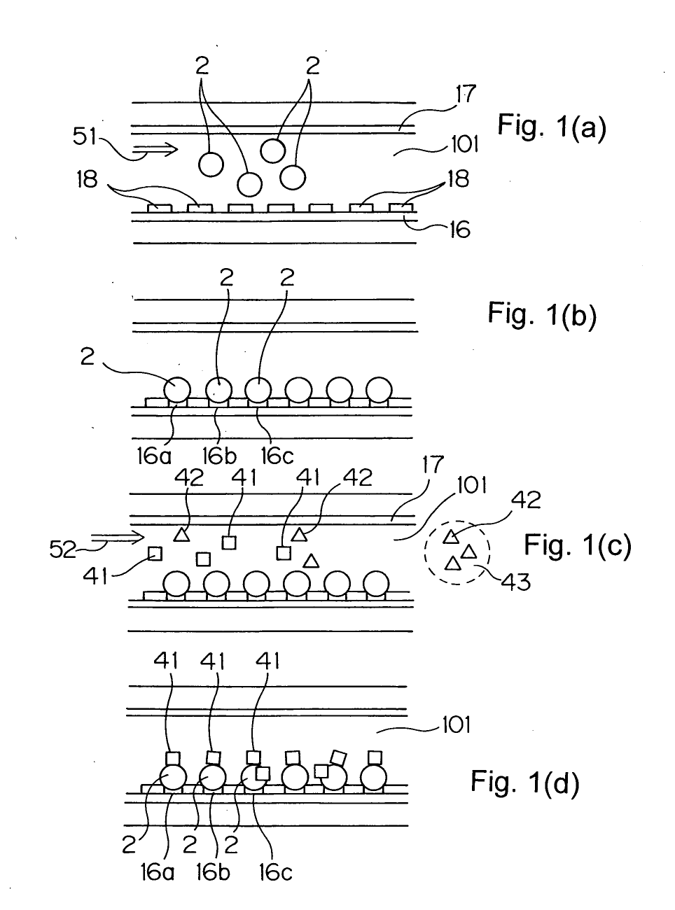

Referring to FIG. 1, a description shall be provided for the cell accumulation method of the present invention. The cell accumulation method comprises the bead trapping process and the cell collection process that follows the bead trapping process. Each of the figures of 1(a) to 1(d) is a cross-sectional view of the accumulation and cultivation vessel. (a) shows the microbead trapping process of in progress, and (b) shows the microbead trapping process that came to an end. (c) shows the cell collecting process of cells in progress and (d) shows the cell collecting process that came to an end.

In the microbead trapping process, while the suspension of the collagen microbeads 2 is sent to the inside of the vessel 101 of the accumulation and cultivation vessel 10 from the inlet 14, AC voltage is applied to the first electrode unit 16 and the second electrode unit 17 by the power supply unit 30. In FIG. 1(a), the arrow 51 shows the flow direction of the suspension of the collagen microbeads 2.

Because the first electrode unit 16 comprises the individual electrodes 16a, 16b, 16c, . . . , which have a microscopic surface and because the second electrode unit 17 has a surface stretching out over all of the top surface of the inside of the vessel, an unequal alternating electric field is produced in the inside of the vessel 101. The collagen microbeads 2 are dielectrophoresed and drawn to the first electrode unit, because alginate was added to the collagen microbeads 2.

Referring to FIG. 1(b), one (1) collagen microbead 2 is trapped on each of the individual electrodes 16a, 16b, 16c, after the microbead trapping process. In the cell cultivation after that, the collagen microbead becomes a scaffold material for cultivating the cells. It is preferable that one collagen microbead be trapped on one individual electrode by adjusting the flow rate of the microbead suspension, frequency and voltage of the applied alternate current, the particle size of the microbeads, the distance between individual electrodes, etc. in the microbead trapping process, because these arrangements allow the cultivation in a single accumulation and cultivation vessel 10 to be homogenized at the time of cell cultivation. For example, the use of beads with larger diameter than that of the individual electrode is thought to be appropriate for one bead to be trapped on one individual electrode.

In the cell collection process, while the cellular suspension is sent to the inside of the vessel 101 of the accumulation and cultivation vessel from the inlet 14 after end of the process of trapping the collagen microbeads, AC voltage is applied to the first electrode unit 16 and the second electrode unit 17 by the power supply unit 30. Described as an example here is a separation model, for which active cells 41, that are cells targeted for collection, and simulated inactive cells 42 coexist in the cellular suspension.

Referring to FIG. 1(c), the arrow 52 shows the flow direction of the cellular suspension. The behavior of the active cells 41 is different in dielectrophoresis from that of the simulated inactive cells 42. Therefore, if the appropriate frequency and voltage are selected, the active cells 41 are collected on the collagen microbeads 2, while the simulated inactive cells 42 drain from the outlet 15 with the flow of the cellular suspension. The wavy line 43 in the figure shows the draining solution.

Referring to FIG. 1(d), after the end of the cell collection process, the collagen microbeads 2, on which the active cells 41 were collected, are trapped on the individual electrodes 16a, 16b, 16c, . . . in the inside of the vessel 101 of the accumulation and cultivation vessel 10. The figure, in which one (1) or two (2) active cell(s) 41 is(are) collected on one collagen microbead 2, is simplified for the purpose of easy explanation of the present invention. In the actual test, two (2) or more active cells 41 were collected on one (1) collagen bead.

Explained above was the cell collection process in the separation model. The cell collection process of the present invention may concentrate the collection target cells. Next, a description shall be provided for the cell collection process, taking a concentration model, where only the active cells 41 exist in the cellular suspension, as an example.

In the cell collection process, while the cellular suspension is sent to the inside of the vessel 101 of the accumulation and cultivation vessel from the inlet 14 after the end of the process of trapping the collagen microbeads, AC voltage is applied to the first electrode unit 16 and the second electrode unit 17 by the power supply unit 30. If the appropriate frequency and voltage are selected, the active cells 41 are collected on the collagen microbeads 2.

After the end of the cell collection process as described above, the culture solution is put into the inside of the vessel 101 of the accumulation and cultivation vessel 10, where the culture is then performed.

When the cells grow, the whole surface of the collagen microbeads are covered by the growing cells. When the cultivation continues further, the cells absorb the collagen microbeads. Such growing cells are expected to mass in a spherical shape.

Embodiment of the accumulation and cultivation vessel of the present invention, which is followed by the cultivation, does not require work which is necessary for sterilization of test tubes, petri dishes, and other tools needed for conventional separation and cultivation.

### Embodiment 1

—Separation Model—

The accumulation and cultivation vessel was prepared by using silicon gaskets as sidewall material and by setting glasses with ITO thin film on the upper and down sides thereof, which glasses were pressed. The first electrode unit was made by photolithography, for which thick resist SU-8 was used.

The silicon gasket was 500 μm thick. The inside of the vessel was 15 mm long and 15 mm wide, and had a volume of 112.5 mm³. The individual electrodes, which were circular, were allocated at each corner of latticed squares with a distance of 550 μm between lattices. Two (2) kinds of electrode were manufactured: one was 50 μm in diameter and the other was 100 μm in diameter.

The collagen microbeads, which are a mixture of collagen and alginate as described above, were manufactured by the method described above. The diameter of the collagen microbeads ranged between 70 and 120 μm, with a median of approximately 100 μm.

Cartilage cells of knee joints of 1- to 2-months-old calves were used as active cell and plastic fine particles (Polybead Pholystyrene Microspheres (2,5% Solids-Latex) 10 μm manufactured by Polysciences, Inc.) were used as simulated inactive cell. The plastic fine particles were approximately 10 μm in diameter.

Low conductive physiological buffer solution, that was cell isotonic solution, was used as solvent of the collagen microbead suspension and the cellular suspension. The collagen microbead suspension was prepared to have a density of 0.5 to 1.0×10⁵ pieces/ml. For the cellular suspension, the active cells were prepared to have a cell density of 2.0×10⁶ cells/ml and the simulated inactive cells were prepared to have a cell density of 2.5 to 5.0×10⁶ cells/ml.

The trapping condition of the collagen microbeads was microscopically observed by using Collargen Stain Kit manufactured by COSMO BIO Co., Ltd., in which case the collagen microbeads were dyed red.

After the end of the cell collection process, the accumulation and cultivation vessel was kept at 27° C. by using a silicone rubber heater to set cartilage cells on the collagen microbeads. After that, the inside of the vessel was filled with a feed medium (DMEM/F12, 20% FBS, Antimycotic-Antibiotic) to cultivate the cells for one (1) week. The culture environment was at a temperature of 37° C., at 5% CO₂, and at 100% humidity.

The situation of the cell collection after the end of the cell collection process and the cells after the end of the cultivation were observed by using a microscope.

### <Microbead Trapping Process>

The AC voltage applied was 25V, which was a voltage between peaks of sine-wave AC voltage (that is to say, amplitude×2 was 25 V). Hereinafter, the voltage applied is shown “OO Vp−p.” 00 is a numerical value showing a voltage between peaks.

The experiment was performed at an applied frequency of 500 kHz and at flow rates of 0.25 ml/min, 0.5 ml/min, and 1.0 ml/min of the collagen microbead suspension. The microbeads were trapped only on the individual electrodes at flow rates of 0.5 ml/min and 1.0 ml/min, while at flow rates of 0.25 ml/min the microbeads precipitated and were trapped both on the individual electrodes and on the insulated layer areas.

When the accumulation and cultivation vessel with the individual electrodes of 50 μm in diameter was used at a flow rate of 1.0 ml/min of the microbead suspension, one (1) microbead was trapped on each of the individual electrodes. On the other hand, when the diameter of the individual electrodes was 100 μm, two (2) or more microbeads were trapped on each of the individual electrodes. The result shows that the use of the microbeads having greater diameter than that of the individual electrodes is suitable for one (1) microbead to be trapped on each of the individual electrodes.

Furthermore, the experiment was performed at an applied frequency of 1 MHz. There was little difference in trapping of the microbeads between 500 kHz and 1 MHz.

### <Cell Collection Process>

The experiment was performed at an applied voltage of 20 Vp−p, at applied frequencies of 500 kHz and 1 MHz, and at flow rates of 0.10 ml/min, 0.25 ml/min, 0.50 ml/min, and 1.0 ml/min.

When the flow rate was 0.25 ml/min, the largest number of cells accumulated on the microbeads. On the other hand, when the flow rate was 1.0 ml/min, the cells did not accumulate on the microbeads; therefore, they flown away. There was little difference in collecting of cells between 500 kHz and 1 MHz.

### <Cell Cultivation>

The cartilage cells that had scattered and adhered to the surface of the collagen microbeads after the end of the cell collection process, stayed on the collagen microbeads and accumulated after the cultivation.

### Embodiment 2

### <Concentration Model>

The accumulation and cultivation vessel used for Embodiment 2 was the same as that for Embodiment 1. Cartilage cells of knee joints of 1- to 2-months-old calves were used. Low conductive physiological buffer solution, that was cell isotonic solution, was used as solvent of the cellular suspension. The cellular suspension was prepared to have a cell density of 2.0×10⁶ cells/ml. Unlike Embodiment 1, the cellular suspension did not contain simulated inactive cells.

### <Microbead Trapping Process>

Because the microbead trapping process in this embodiment is the same as that described in Embodiment 1, the explanation is omitted.

### <Cell Collection Process>

The experiment was performed at an applied voltage of 20 Vp−p, at applied frequencies of 500 kHz and 1 MHz, and at flow rates of 0.10 ml/min, 0.25 ml/min, 0.50 ml/min, and 1.0 ml/min.

When the flow rate was 0.25 ml/min, the largest number of cells accumulated on the microbeads. On the other hand, when the flow rate was 1.0 ml/min, the cells did not accumulate on the microbeads; therefore, they flown away. There was little difference in collecting of cells between 500 kHz and 1 MHz.

### <Cell Cultivation>

The cartilage cells that had had scattered and adhered to the surface of the collagen microbeads after the end of the cell collection process, were set on the collagen microbeads and accumulated after the cultivation. The cultivation conditions were the same as those for Embodiment 1.

### DESCRIPTION OF THE REFERENCE NUMERAL

1 Cell collection device
2 Collagen bead (bead having biocompatibility)
10 Accumulation and cultivation vessel
11 First substrate
12 Second substrate
13 Sidewall material
14 Inlet
15 Outlet
16 First electrode unit
16a, 16b, 16c Individual electrodes
17 Second electrode
18 Insulating photo-resist film
20 Bead making vessel
21 Material pushing out nozzle
22 Gas nozzle
23 Gelling solution
30 Power supply unit
40 Cellular suspension
41 Active cell
42 Simulated inactive cell
43 Outflowing solution
101 Inside of the vessel
161 Terminal
171 Terminal

What is claimed is:

1. A cell accumulation method comprising
a cell accumulation device comprising
an accumulation and cultivation vessel with an inlet and an outlet, surrounded by a first substrate, a second substrate allocated against the first substrate, and the sidewall, having a first electrode unit, composed of the plural number of individual electrodes exposed on the inside of the accumulation and cultivation vessel, allocated on the first substrate and a second electrode unit allocated on the second substrate, and
a power supply unit applying AC voltage between the first electrode and the second electrode, and
a cell accumulation method comprising the following processes a) and b) which method utilizes dielectrophoretic micro microbeads having biocompatibility;
a) a microbead trapping process, where a microbead having biocompatibility is sent to said accumulation and cultivation vessel from said inlet and AC voltage is applied to the first electrode unit and the second electrode unit by said power supply unit to trap said microbeads on the individual electrodes; and
b) a cell collection process, where after the microbead trapping process cellular suspension containing collection target cells is sent into said accumulation and cultivation vessel from said inlet and AC voltage is applied to the first electrode unit and the second electrode unit by said power supply unit to collect said collection target cells on said trapped microbeads.

2. A cell accumulation method according to claim 1, wherein said microbead having biocompatibility is a bead comprising collagen and alginate.

3. A cell accumulation method according to claim 1, wherein the first electrode unit has said individual electrodes, where an electrode member having a flat surface shape is formed on the surface of the first substrate, an insulated layer comprising insulating material is formed on the surface of said electrode member, and said insulated layer is removed partly in a circle.

4. A cell accumulation method according to claim 3, wherein the first electrode unit has said individual electrodes, where a conducting layer of said electrode member is formed on the surface of the first substrate, said insulated layer is formed on said conducting layer, and said insulated layer of said individual electrode part is removed by irradiation of a laser, by using the laser-etching method.
